(19) United States
(12) Patent Application Publication
(10) Pub. No.: US 2011/0308531 A1
(43) Pub. Date: Dec. 22, 2011

(54) Adjustable oral appliance for treatment of snoring and sleep apnea

(75) Inventor: Phillip P. Grosky, Anaheim, CA (US)

(73) Assignee: James R. Glidewell Dental Ceramics, Inc.

(21) Appl. No.: 13/134,359

(22) Filed: Jun. 6, 2011

Related U.S. Application Data
(60) Provisional application No. 61/397,932, filed on Jun. 18, 2010.

(51) Int. Cl. A61F 5/56

(52) U.S. Cl. 128/848

(57) ABSTRACT

A mandibular advancement oral appliance for treatment of snoring and obstructive sleep apnea comprises upper and lower thermoformed co-polyester splints, custom fabricated to match the patient's dentition. The splints are interconnected by a thread cord or filament in a pre-fabricated continuous loop with no knot. The loop passes through guide tubes and around pulley's having openings to allow the thread to be forced into them. The thread is made up of braided liquid crystal polymer fiber (Vectran®). This appliance uses a locking device to keep the continuous loop from traveling along itself. This stabilizes the anterior positioning. The locking mechanism comprises an expansion clamp consisting of an oval expansion crank, oval casing and cap. The locking mechanism's function is to restrict movement of the loop, thereby locking the mandible in the desired forward location while the patient sleeps. The loop guide, pulleys and locking device are preferably affixed to the anterior-facing surfaces of the splints.

### CROSS-REFERENCE TO RELATED APPLICATIONS

This application takes priority from provisional application Ser. No. 61/397,932 filed on Jun. 18, 2010.

### BACKGROUND OF THE INVENTION

1. Field of the Invention

The present invention relates to an oral appliance configured to pull and secure the mandible forward to better open the airway and allow freedom of air flow. This Mandibular Repositioning (MRP) device is indicated to mitigate snoring or to treat obstructive sleep apnea (OSA).

2. Background Art

It has been estimated that more than 6 million adults (with men outnumbering women 8 to 1) snore and that a high percentage of those people may have sleep apnea. MRP devices may be the appropriate treatment for persons with mild to moderate sleep apnea when surgical and other medical treatments are ineffective or not desired. Such devices are preferably adjustable to permit effective air flow without incurring discomfort or even pain.

Other devices of this nature (MRP's) are often designed with some sort of strap system to pull the mandible forward. These appliances have too many variations of straps and require changing of the straps on a regular basis. Other alternative designs include metal parts and screws for mechanically advancing the mandible. These require tools to adjust or operate and often limit the lateral movement of the mandible, creating discomfort on the TMJ. Also, these metal parts can incur an unpleasant taste of the appliance and can often incur problems with oxidation. The appliance herein is designed to be metal free and employs a tensile thread cord or filament which has a very high tensile strength. There are no straps to change or of which to keep track. There are no metal parts to rust. No tools are required to accomplish mandibular advancement. One simply releases a locking device, slides the mandible forward, either by pushing the jaw forward or by pulling with a finger or dental instrument and then re-locks the appliance.

### SUMMARY OF THE INVENTION

A mandibular advancement oral appliance for treatment of snoring and obstructive sleep apnea comprises upper and lower thermoformed co-polyester splints, custom fabricated to match the patient's dentition. Other materials can be utilized for the splints, including but not limited to, poly methyl methacrylate (PMMA), and ethyl vinyl acetate (EVA).

The splints are interconnected by a thread cord or filament in a pre-fabricated continuous loop with no knot. The loop passes through guide tubes and around pulley's having openings to allow thread to be forced into them. In a preferred embodiment, the thread is made up of braided liquid crystal polymer fiber (Vectran®). This appliance uses a twist-locking device to keep the continuous loop from traveling along itself. This stabilizes the anterior positioning of the lower splint and prevents movement thereof during sleep.

The locking mechanism is an oval expansion clamp consisting of an oval crank, oval casing and lid. The thread travels within the casing. Alignment of the oval shapes creates a clamping force on the braided poly loop. Misalignment of the oval mechanism allows free movement of the loop. Material for tension lock can be of any biocompatible material. Methods of restricting movement of loop may be accomplished by other means, including, but not limited to, various clamping methods or locking spools. Prior patents such as U.S. Pat. No. 6,325,064, require a permanent fixture of the tensile member. The present invention has no fixed anchoring of the thread/tensile member. The locking mechanism's function is to restrict movement of the loop, thereby locking the mandible in the desired forward position. It should be noted that the movement of the lower mandibular portion in relation to the upper, actually defines the location at which the loop holds the device. There is virtually no lateral excursion limitation due to the routing of the tensile member through the loop guide and pulleys. Loops are available in various lengths to accommodate different sized patient arches. Guidance can be accomplished by varying means such as solid tubes or fixed position parts with holes for thread to slide through.

The advancement of the mandible is initiated by the release of the locking mechanism and the sliding of the patient's mandible in either direction (forward/backward). The tensile thread slides through the locking mechanism when the two ovals are misaligned. As soon as the ovals are aligned, the expansion clamp stops the thread from moving, thereby locking the mandible in its position. The thread or loop can be removed from pulleys and guide tubes to allow occlusal or lingual adjustments to the splints with acrylic. Removing the end of the loop that travels from the upper posterior to the lower loop guides allows the appliance to be opened fully to add any orthodontic acrylic resins for vertical adjustment. Also, by removing the lid of the locking mechanism, the entire thread can be replaced without having to replace guide tubes or pulleys.

Each pulley is a solid piece of plastic designed to allow the loop to slide through a specific location on the splint. Each pulley specifically allows the loop to be held within the pulley, but also allows the loop to be removed from the appliance at will. The inhibitor for the loop is an extension at the exit to the pulley that makes the space too small for the loop to exit on its own slack. The invention may include other methods for changing the route of the continuous loop.

### BRIEF DESCRIPTION OF THE DRAWINGS

The aforementioned objects and advantages of the present invention, as well as additional objects and advantages thereof, will be more fully understood herein after as a result of a detailed description of a preferred embodiment when taken in conjunction with the following drawings in which:

FIG. 1A is a three-dimensional view of the preferred embodiment of the MRP device of the present invention;

FIG. 1B is a view of a continuous loop thread use in the preferred embodiment;

FIG. 2 comprising FIGS. 2A, 2B, 2C and 2D are top, side, front and three-dimensional views, respectively of a loop guide used in the preferred embodiment;

FIG. 3, comprising FIGS. 3A, 3B and 3C are top, profile and front views, respectively of a pulley used in the device;

FIG. 4 is a top view of the locking mechanism used in the illustrated embodiments shown in its locked configuration;

FIG. 5 is a view of the locking mechanism of FIG. 4, but shown in its unlocked configuration;

FIG. 6 is an exploded top view of the locking mechanism;

FIG. 7 is an exploded bottom view of the locking mechanism; and

FIG. 8 is an elevational front view of the MRP device shown fully assembled.

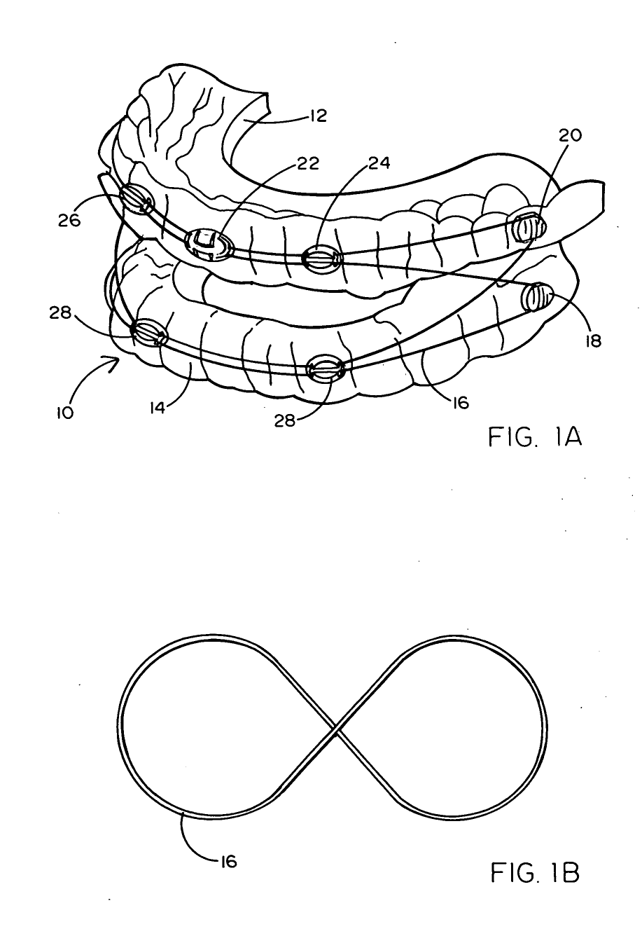

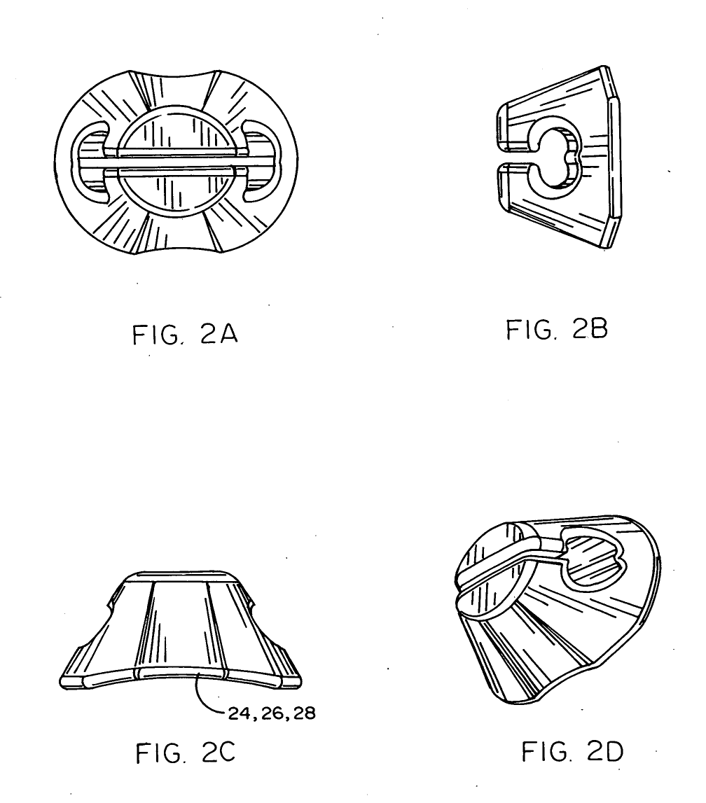

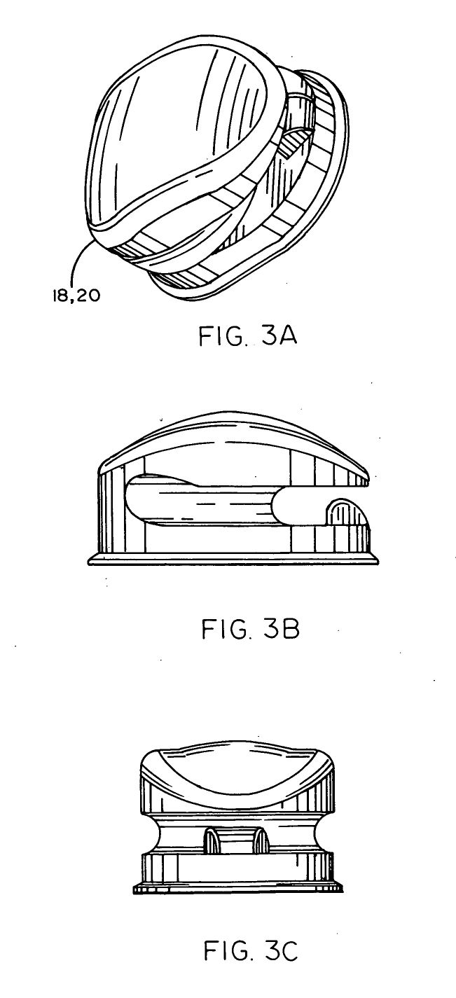

### DETAILED DESCRIPTION OF A PREFERRED EMBODIMENT

Referring now to the accompanying figures, it will be seen that the adjustable oral appliance 10 of the present invention comprises an upper splint 12 and a lower splint 14 interconnected by a continuous loop thread 16. The splints 12 and 14 are configured to be geometrically compatible with a patient's dentition while forcing the mandible forward to open the airway to reduce or prevent snoring and sleep apnea.

The thread 16 is routed around a pair of lower pulleys 18 and a pair of upper pulleys 20, through upper loop guides 24 and 26 and lower loop guides 28.

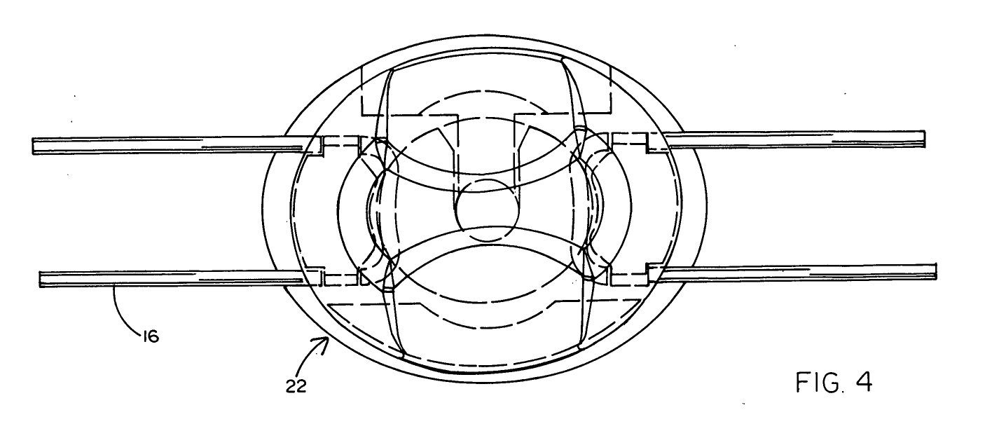

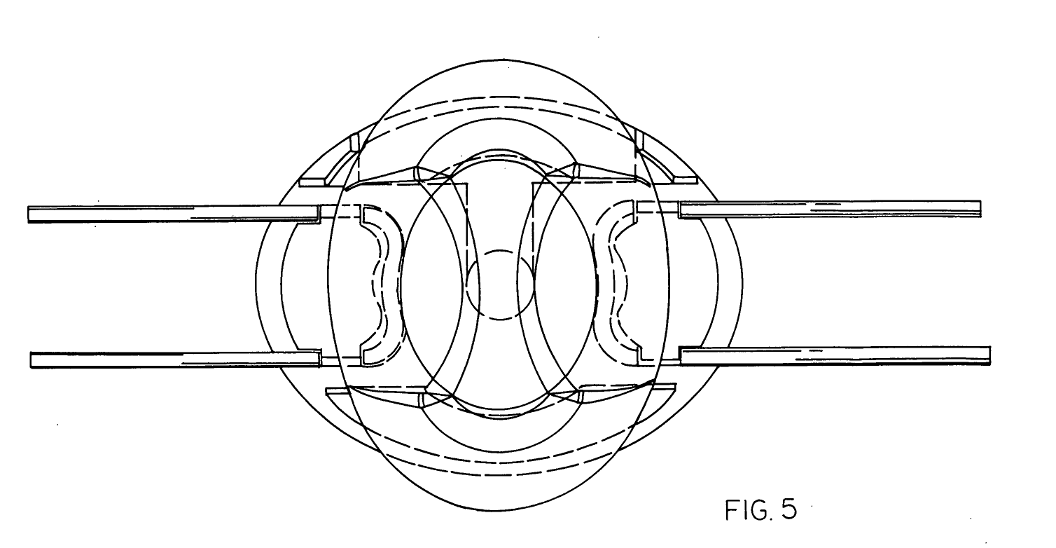

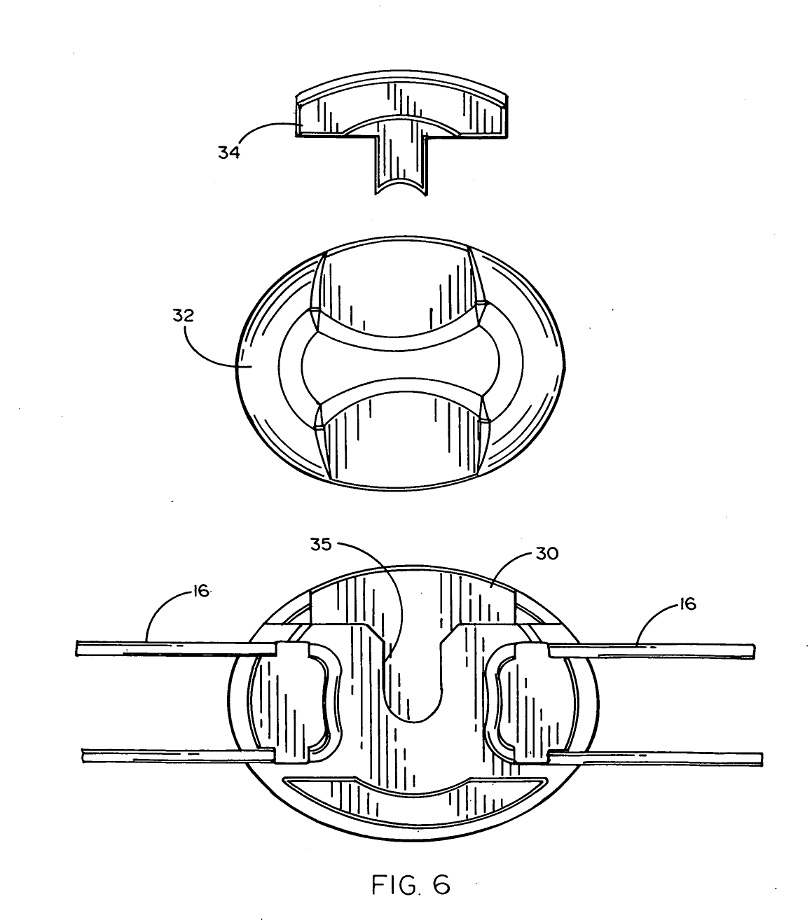

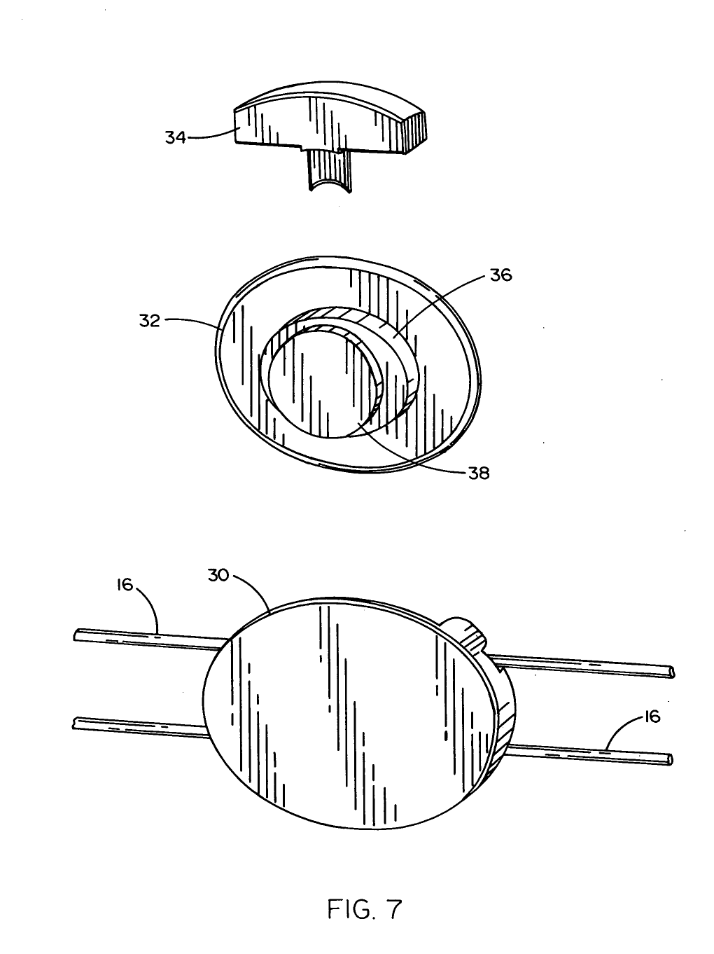

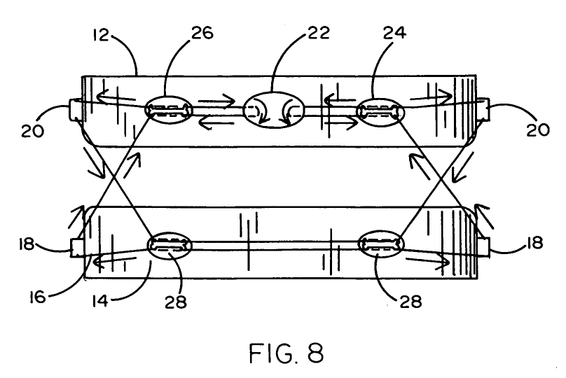

Adjustment of the appliance, which consists of moving the lower splint forward or backward relative to the stationary upper splint, requires release of a locking mechanism 22 which is mounted on the upper splint centered between the upper loop guides. The continuous loop thread 16 is twice routed through the locking mechanism, the components of which are shown in FIG. 4 through FIG. 7.

As seen therein, locking mechanism 22 comprises a base 30, an insert 32 and a cap 34. Insert 32 has an oval 36 and a wedge 38. Base 30 has a recess 35 shaped to receive wedge 38. Base 20 receives two opposed loops of thread 16. When insert 32 is inserted into the base so that the wedge 38 is captured in recess 35, oval 36 will either functionally engage the loops of thread 16 to lock the thread or disengage to allow movement or slippage of thread 16. Insert 32 is rotated to lock or unlock the thread. Cap 34 is used to prevent inadvertent rotation of the insert.

The method of routing the thread is used to accomplish both the simplicity of the design and the unique advancement technique for the mandible. In the preferred routing method, a technician performs the following steps:

1. The loop is slid into the lower anterior loop guides.

2. The loop is routed to the pulleys on each corresponding side of the appliance.

3. Next, the thread is routed to the upper anterior loop guide on each side.

4. Each side of the loop is routed through the mounted oval casing and exited out of the same side of the oval.

5. The loop is then routed back to the upper anterior loop guides.

6. The oval expansion crank is placed into the locking mechanism, and then the cap is positioned. There should be two portions of the loop running through each upper loop guide at this point.

7. Finally, the loop is routed to the lower anterior loop guides on either side. Finish by routing the two free ends to the upper posterior pulleys. This method can be varied accordingly.

Routing can also be finished by placing a loop end into the lower guide tube and placing the upper pulleys on the loop on each side, then placing each pulley on either side of the upper posterior. Slack in the loop is pulled towards the rear of the appliance and secured using light cure, or any other method of attaching to co-polyester/plastic splints.

This entire process can be done with all routing components mounted in various locations, including reversal of upper and lower mechanisms. The locking mechanism is not limited to any particular location, as long as it prohibits further sliding of the loop. However, center anterior-facing is probably the most convenient for adjustment and locking.

It will now be understood that what has been described herein is an adjustable oral appliance for treating patients for snoring and/or sleep apnea. The appliance employs a unique continuous loop thread cord or filament and a pair of dentition splints to overcome disadvantages of prior art attempts to address these problems. It will be also understood that the scope hereof is not limited to the disclosed embodiment which is provided as a best mode example of the preferred configuration. Only the appended claims define the metes and bounds of the invention herein disclosed.

I claim:

1. An adjustable oral appliance for moving and locking a patient's mandible in a forward position and thereby further opening the patient's airway to mitigate snoring and sleep apnea; the appliance comprising:
upper and lower splints configured to mate with the patient's dentition; and
a continuous loop thread routed through components attached to anterior-facing surfaces of both said upper and lower splints;
one of said components being configured as a locking mechanism for selectively allowing and preventing slippage of said thread to permit moving adjustment of said lower splint relative to said upper splint and then locking said splints to prevent further adjustment while the patient sleeps.

2. The oral appliance recited in claim 1 wherein at least one of said components comprises a loop guide for routing said loop thread laterally along said anterior-facing surfaces.

3. The oral appliance recited in claim 1 wherein at least one of said components comprises a pulley for routing said loop thread between said upper and lower splints.

4. The oral appliance recited in claim 1 wherein said locking mechanism comprises at least one adjustable device for compressively engaging said loop thread for locking said splints in a first orientation and releasing said loop thread for allowing slippage of said loop thread in a second orientation.

5. An oral appliance for locking patient's mandible in a forward thrusted position to further open the patient's airway during sleep; the appliance comprising:
upper and lower dentition-impressed splints to mate with the patient's teeth in opposed relation;
a plurality of routing components affixed to surfaces of the respective splints;
a continuous loop cord routed through said routing components of both said splints to attach the upper and lower splints in an adjustable configuration permitting limited selective movement of said lower splint relative to said upper splint while they remain attached to each other.

6. The oral appliance recited in claim 5 wherein at least one of said routing components comprises a locking mechanism for selecting locking of said cord to prevent further movement of said lower splint.

7. The oral appliance recited in claim 5 wherein at least one of said components comprises a loop guide for routing said loop cord laterally along said anterior-facing surfaces.

8. The oral appliance recited in claim 5 wherein at least one of said components comprises a pulley for routing said loop cord between said upper and lower splints.

9. The oral appliance recited in claim 6 wherein said locking mechanism comprises at least one adjustable device for compressively engaging said loop cord for locking said splints in a first orientation and releasing said loop cord for allowing slippage of said loop cord in a second orientation.

10. A method for adjusting a patient's mandible forward to open the patient's airway to mitigate snoring and sleep apnea; the method comprising the steps of:
forming upper and lower dentition impressed splints to mate with the patient's upper and lower dentition;
affixing anterior-facing routing components to said splints'
installing a continuous loop thread into said routing components to bind said splints to each other in adjustable engagement;
configuring at least one of said components as a locking mechanism selectively allowing and preventing slippage of said thread to permit movement adjustment of said lower splint relative to said upper splint and then locking said splints to prevent further adjustment.
